(19) United States
(12) Patent Application Publication
(10) Pub. No.: US 2021/0015665 A1
(43) Pub. Date: Jan. 21, 2021

(54) SHUNT IMPLANT

(71) Applicant: Carl Zeiss Meditec AG, Jena (DE)

(72) Inventors: Martin HACKER, Jena (DE); Rudolf Murai von Bünau, Jena (DE); Christian DIETRICH, Jena (DE)

(73) Assignee: Carl Zeiss Meditec AG, Jena (DE)

(21) Appl. No.: 16/978,568

(22) PCT Filed: Mar. 6, 2019
(86) PCT No.: PCT/EP2019/055579

§ 371 Date: Sep. 4, 2020

(30) Foreign Application Priority Data
Mar. 7, 2018 (DE) 10 2018 203 424.9

(51) Int. Cl. A61F 9/007

(52) U.S. Cl. CPC A61F 9/00781; A61F 2220/0041; A61F 2250/0091; A61F 2230/0006; A61F 2230/0008

(57) ABSTRACT

A drainage aid which is introduced into tissue to permit or improve drainage of liquid by generating new drainage channels or by keeping existing drainage channels open, to permit effective drainage. In particular, a stent for glaucoma treatment is provided for the drainage of aqueous humor from the anterior chamber through the cornea, the limbus or the sclera directly onto the eye surface. The shunt implant includes at least one inner component and one outer component, which are connected to each other following introduction. The cross section of the shunt implant can be round, oval or angular. The invention can also be used wherever narrowed vessels or channels are intended to be expanded or held open.

### RELATED APPLICATIONS

This application is a National Phase entry of PCT Application No PCT/EP2019/055579 filed Mar. 6, 2019, which application claims the benefit of priority to DE Application No. 10 2018 203 424.9 filed Mar. 7, 2018, the entire disclosures of which are incorporated herein by reference.

### TECHNICAL FIELD

The present invention relates to a drainage aid which is introduced into tissue in order to permit or improve drainage of liquid.

### BACKGROUND

Such implants for drainage of liquid are well known in the prior art. While stents are usually understood as drainage aids for opening or for keeping open vessels or tissues, shunts serve as drainage aids for bridging or bypassing natural drainage paths. However, these functions can also be applied simultaneously or can overlap. According to the invention, the term shunt used below can comprise both functions.

### SUMMARY OF THE INVENTION

In the present case, the implant therefore serves the purpose of generating new drainage channels or keeping existing drainage channels open, in order to permit effective drainage. In particular, the proposed implant is provided for glaucoma treatment for the drainage of aqueous humor from the anterior chamber through the cornea, the limbus or the sclera directly onto the eye surface, hereinafter referred to as direct drainage. The implants can remain permanently in the body as drainage aids or they can also be made partially or completely from bioresorbable substances, for example with pharmaceutical action.

Glaucoma is understood as the excavation of the optic nerve Progressive damage to the optic nerve causes a continuous decrease in the field of vision of the patient. Without treatment, this in most cases leads to complete loss of sight.

The precise cause of glaucoma or of the described damage to the optic nerve is not fully understood at present. However, the most likely trigger has been identified as being an increase in intraocular pressure caused by deterioration in the drainage of aqueous humor. As a consequence of this deterioration in the drainage of aqueous humor, i.e. the increased drainage resistance, the pressure within the eye builds up until, with the intraocular pressure now increased, the drainage of aqueous humor is once again in equilibrium with the production of aqueous humor.

A deterioration in the drainage of aqueous humor can be caused, for example, by a narrowing of the iridocorneal angle (narrow-angle glaucoma) or also, in the case of open-angle glaucoma, by changes to the filter tissue of the trabecular meshwork or by complete blockage thereof (for example in the case of pseudoexfoliation glaucoma or pigmentary glaucoma), or also as a result of a reduction in the cross section of Schlemm's canal or of downstream collector vessels or in the episcleral vascular system. Changes to tissues in the uveoscleral outflow pathway may also lead to deterioration in the drainage of aqueous humor.

In narrow-angle glaucoma, the intraocular pressure is in most cases lowered directly by surgical interventions, for example iridectomy.

In pseudoexfoliation glaucoma and pigmentary glaucoma, the drainage of aqueous humor is often improved by performing aspiration during an intervention.

In the treatment of open-angle glaucoma, a reduction in the intraocular pressure is in most cases sought in the first instance by medication, i.e. using substances which either reduce the production of aqueous humor (e.g. beta blockers) or which improve the flow through the tissues of the outflow pathways (e.g. prostaglandins).

In addition, an improvement in the trabecular outflow can also be obtained through laser trabeculoplasty procedures (selective laser trabeculoplasty (SLT), argon laser trabeculoplasty (ALT), excimer laser trabeculoplasty (ELT)).

Moreover, canaloplasty procedures are known in which Schlemm's canal is widened. If the glaucoma worsens, a trabeculotomy (partial excision of the trabecular meshwork) or a partial coagulation of the aqueous-humor-producing ciliary body is considered, for example in the form of cyclophotocoagulation (CPC), cyclocryocoagulation (CRC) or ultrasonic cyclocoagulation (UCC).

If these measures cannot be carried out or are inadequate, recourse is in most cases made to filtration surgery, for example in the form of trabeculectomy, in which an artificial outflow path into a bleb is formed under the connective tissue.

For the treatment of advanced glaucoma, trabeculectomy is considered the standard procedure, although the rate of complications resulting from reactions by the body (scarring) is relatively high, the scarring process has to be controlled by intraoperative use of antifibrotic agents and, in many cases, follow-up measures (needling) are nevertheless required.

In addition, larger implants (tube shunts) are used in which aqueous humor is conveyed through a hose to a drainage plate which is secured on the sclera, but underneath connective tissue, in most cases by suturing, and from which the aqueous humor is conveyed into a bleb under the eye surface (Baerveldt implant inter alia). These implants can additionally have valve effects too (Ahmed valve).

In the prior art, other surgical forms of glaucoma treatment with reduced invasiveness have also become known in recent years, these being intended to have a greater potential for pressure reduction along with reduced rates of complications, for example through the use of minimally invasive stents and shunts (for example for bridging the trabecular meshwork and for keeping open Schlemm's canal (iStent, HYDRUS) or also for drainage into the supraciliary space (CYPASS) or into the subconjunctival space (XEN, MicroShunt). Stents or shunts can also be constructed from porous material and can also be designed to be controllable according to the intraocular pressure (U.S. Pat. No. 8,926,510 B2). The abovementioned surgical interventions are classed as ab interno or ab externo interventions, depending on whether the manipulation or also the implantation is performed from inside the eye or from outside the eye. For example, canaloplasty procedures can be performed as ab interno interventions or also as ab externo interventions. Examples of drainage aids that can be implanted from inside the eye are iStent, HYRUS, CYPASS and XEN, while the MicroShunt is an example of an insertion aid that can be implanted from outside the eye.

Reference is also made for example to U.S. Pat. No. 6,881,198 B2 and U.S. Pat. No. 3,788,327 A, which describe corresponding surgical implants for lowering the intraocular pressure by drainage of excess aqueous humor. These surgical implants in the form of stents use direct drainage through the cornea, the limbus or the sclera. The stents furthermore comprise a filter membrane in order to ensure a defined outflow.

Devices for treating glaucoma are also described in WO 2016/109639 A2, although the focus there is on additional measures for secure anchoring of such stents in the tissue.

A disadvantage of the solutions mentioned is that the structures for retention of the described stents place a very great strain locally on the tissue and pose the danger of tearing out. For removal or replacement, the retention structures have to be overcome, which results in extreme strain on the tissue and possible damage during the removal or replacement.

### SUMMARY OF THE INVENTION

Embodiments of a shunt implant overcome many of the disadvantages of the known solutions. In particular, the invention is intended to be such that removal or replacement is carried out without any problems, i.e. without any great strain on the tissue, but while at the same time ensuring a secure hold of the implant.

According to example embodiments of the invention, with the shunt implant for glaucoma treatment for direct drainage through the cornea, the limbus or the sclera, this object is achieved by the fact that the shunt implant is composed of at least one inner component and one outer component, which are connected to each other following introduction. The outer component is inserted and/or manipulated from outside the eye, i.e. ab externo, and the inner component from inside the eye, e.g. ab interno. This means that the solution according to the invention permits minimally invasive glaucoma therapy through a combined ab interno/ab externo intervention, which overcomes the known disadvantages of implantations that are performed either from inside or from outside the eye.

Clip mechanisms or screw threads for example are preferred as connections. Alternatively, magnetic connections, plugging, shrink fitting, welding or adhesive bonding are possible further examples. The latter connections can be configured such that they can be undone in the event of removal of the shunt implant (e.g. by breaking them by twisting the parts of the shunt implant relative to each other), without placing an unacceptable strain on the tissue.

According to an example embodiment, the at least one inner component of the shunt implant has a pressure-reducing element, which is designed specific to the patient and/or is designed to be exchangeable.

Advantageously, the components of the shunt implant are present in different dimensions, as a result of which an adaptation to different thicknesses of cornea or sclera can be ensured.

According to a further example embodiment, at least one of the components of the shunt implant has a pressure-measuring means, which can for example be read passively.

The present invention is provided in particular for glaucoma treatment for direct drainage through the cornea, the limbus or the sclera. However, it can also be used wherever narrowed vessels or channels are intended to be expanded or kept open in order to permit effective drainage.

### BRIEF DESCRIPTION OF THE DRAWINGS

The invention is described in more detail below on the basis of exemplary embodiments. Here:

FIG. 1 depicts an inserter with an inner component of the shunt implant,

FIG. 2 depicts an inserter, inserted through a micro-incision on the limbus, for positioning the inner component of the shunt implant,

FIG. 3 depicts a shunt implant according to the invention, composed of an inner component and an outer component, and

FIG. 4 depicts shunt implants according to the invention, in the form of a single implant or also multiple implant.

### DETAILED DESCRIPTION

According to the invention, the shunt implant for glaucoma treatment for direct drainage through the cornea, the limbus or the sclera is composed of at least one inner component and one outer component, which are connected to each other following introduction. The cross section of the shunt implant can be round, oval or else angular. Alternatively, an implant can also be formed from a porous material, such that it has no free internal diameter. It is advantageous if, for example, the implant is made entirely or partially from a material which is transparent in respect of natural and/or artificial ultraviolet light and which has a disinfecting action on and in the implant. A transparency in the range of 390 to 470 nm is particularly advantageous for the deactivation of bacteria and fungi, with at the same time a low cell toxicity for the surrounding tissue, in particular light of about 405 nm wavelength.

According to a first example embodiment, the tool for introducing the at least one inner component of the shunt implant is an inserter through a micro-incision. Micro-incisions are to be understood as small incisions of the kind customary in modern cataract surgery (MICS: micro-incision cataract surgery). These in most cases have incision widths of usually less than 1.8 mm and are configured such that, after removal of the surgical tool, the incisions automatically close again and, without the need for sutures, are sufficiently leaktight. With the aid of a micro-incision, access is also obtained in the prior art to Schlemm's canal, for example, which extends in a ring shape at the iridocorneal angle about the iris. By way of such incisions, it is also possible in the prior art to introduce a microcatheter from the inside into Schlemm's canal in order to widen the latter.

From modern cataract treatment, inserters are known not only for inserting intraocular lenses (IOLs) through micro-inisions but also for implanting minimally invasive glaucoma stents in the trabecular meshwork (iStent G2). Inserters are medical instruments which contain an object that is to be implanted and which can introduce the latter into the target tissue by application of a trigger mechanism.

For this purpose, the inserter is for example inserted through a micro-incision such that the object that is to be implanted is positioned in the target tissue.

Micro-incisions are generated by a lancet, for example. However, it is also possible for this purpose to use an ophthalmological laser therapy appliance. Such ophthalmological laser therapy appliances generally have a laser device with a laser source for generating a pulsed laser beam, a focussing device for focussing the pulsed laser beam at a focus, and a scanning device for scanning the focus of the pulsed laser beam particularly in the cornea, the limbus and/or the sclera of a patient's eye. The tissue in question is modified, microstructured or severed with the aid of the pulsed laser beam.

It is possible in this connection to use a planning device for generating control data for an ophthalmological laser therapy appliance, the latter comprising a laser device with a laser source for generating a pulsed laser beam, a focusing device for focusing the pulsed laser beam on a focus, and a scanning device for scanning the focus of the pulsed laser beam in a tissue of a patient's eye, in particular in a cornea, a limbus and/or a sclera thereof, for modifying, micro-structuring or severing the tissue along a scanning pattern of focal spots of the focus of the pulsed laser beam in accordance with the control data, and a control unit for controlling the ophthalmological laser therapy appliance by means of the control data.

For this purpose, the planning device comprises an interface for supplying data of the characterization of the patient's eye, in particular of the cornea, limbus, and/or sclera of the patient's eye, and for supplying data of a model of a shunt implant for pressure-reducing bridging of the cornea and/or for supplying data of a structure, to be generated in the cornea, the limbus, and/or the sclera, for pressure-reducing bridging of the cornea, and an interface for conveying the control data to a control unit of the ophthalmological laser therapy appliance.

Moreover, such a planning device is designed to generate, from the provided data, control data for the scanning pattern of the focus in a tissue of the patient's eye, in particular in the cornea, the limbus, and/or the sclera, with which control data the ophthalmological laser therapy appliance is controllable in such a way that a structure for pressure-reducing bridging of the cornea can be generated, and/or a structure for receiving the shunt implant for the pressure-reducing bridging of the cornea can be generated.

While the inserter in cataract surgery contains the intraocular lens, in the present case it contains the at least one inner component of the shunt implant, which inner component is positioned via a slide mechanism or spring mechanism.

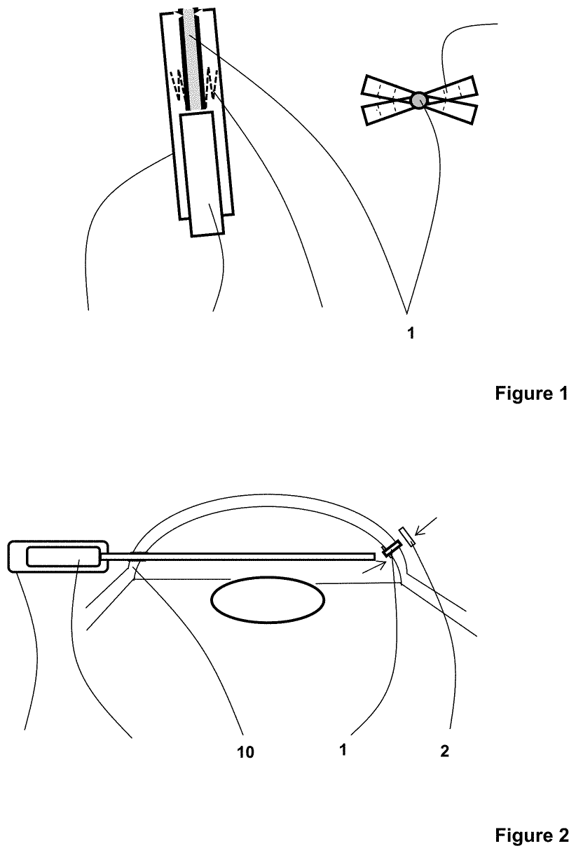

For this purpose, FIG. 1 shows an inserter for insertion of an inner component of the shunt implant, for example through a micro-incision.

The left-hand image shows the inserter 6 with slide mechanism 7 and with the inner component 1 of the shunt implant located in the interior. The inner component 1 has a folding haptic 4, which is shown deployed in the right-hand image.

According to the invention, after insertion of the inserter, for example through a micro-incision on the limbus, at least one inner component of the shunt implant is positioned with the aid of the inserter at a tissue site on the limbus, the cornea or sclera, and a further micro-incision is preferably introduced there. It is particularly expedient if this second microincision lies in the anterior chamber approximately opposite the first micro-incision, since it can then be easily reached with the inserter. This second micro-incision can be opened by a surgical tool from the inside or the outside for the implant that is to be inserted, or else a tissue opening can be produced by a cutting or piercing action of the inserter or of the implant itself.

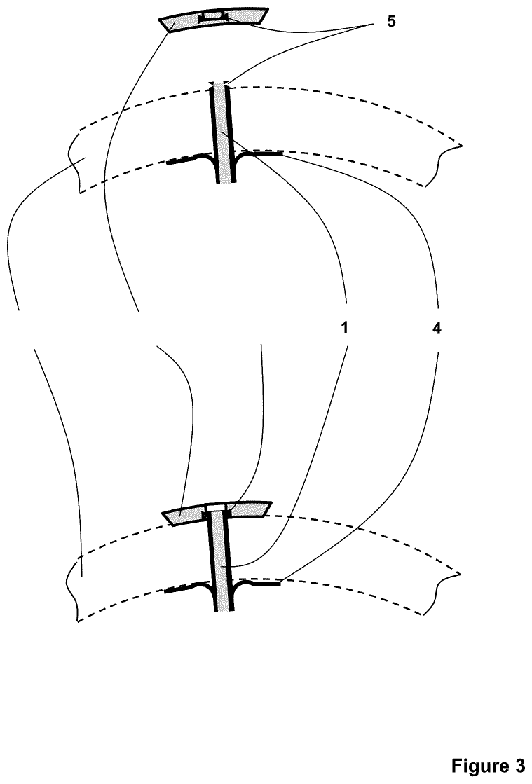

For this purpose, FIG. 2 shows an inserter 6 inserted through a (first) micro-incision on the limbus 10, with a sliding mechanism 7 for positioning the inner component 1 of the shunt implant. In addition to the inserter 6 and the inner component 1 of the shunt implant, the image also shows the outer component 2.

According to a second example embodiment, the at least one inner component of the shunt implant has folding haptics. The folding haptics are kept folded in the inserter until the at least one inner component of the shunt implant is pushed out of the inserter, for example by use of a thrust element, and positioned.

The inwardly folded positioning of the at least one inner component of the shunt implant can be made easier by the fact that the shunt implant has corresponding indentations for the folding haptics.

This allows it to be placed as tightly as possible during the insertion into the inserter. Moreover, the cross section of the inserter can be minimized like the micro-incision required for it.

After implantation, the folding haptics preferably exert a slight spring action on the combined implant, such that the latter is held in place on the cornea. This is particularly advantageous for minimizing friction on the eye surface during eyelid closure. Parts of the inner or outer component, for example the folding haptics, can be of planar configuration in order to permit better distribution of pressure to the tissue, but they can additionally be made permeable to ocular fluids (aqueous humor, tear film liquid) or can have pores or openings in order to cause the least possible disturbance to the metabolism of the contacted tissue.

According to a further example embodiment, the at least one inner component of the shunt implant has a pressure-reducing element which is for example designed specific to the patient and/or is designed to be exchangeable.

The positioning of the pressure-reducing element in the at least one inner component of the shunt implant has the advantage that, even in the case of loss of the at least one outer component, a safe pressure level is maintained.

According to a further example embodiment, the at least one inner component and outer component of the shunt implant are connected by use of a clip mechanism or by a screw thread. Here, the clip mechanism can be designed as an element that is mounted resiliently and with form-fit engagement.

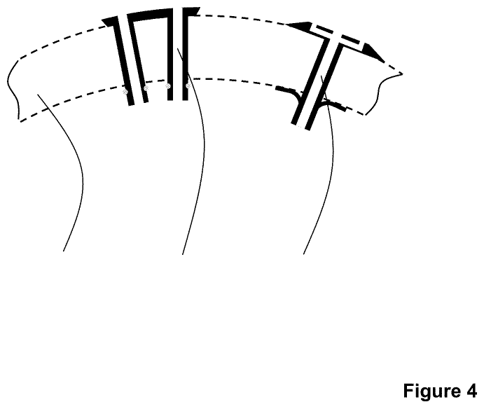

For this purpose, FIG. 3 shows a shunt implant according to the invention, including an inner component and outer component, in the open state and the closed state. Whereas the two components of the shunt implant in the upper image are not yet connected, the lower image shows the two components of the shunt implant when connected.

The shunt implant is composed of an inner component 1 and an outer component 2, which are connected to each other for direct drainage through the cornea 3. As has been explained, the shunt implant can also be applied alternatively on the limbus or also through the sclera. The inner component 1 in this case has folding haptics 4 which conform to the inner wall of the cornea 3, and a slight spring action is thus exerted. The two components 1 and 2 are connected by a clip mechanism 5.

In an example embodiment, the connection between the at least one inner component and the at least outer component of the shunt drainage implant is designed to be reversible, such that the components can be separated again and renewed or replaced.

This has the advantage that the components of the shunt implant can be adapted for example to the course of the pathology or can be removed.

By virtue of the connection of the two components of the shunt implant, the tissue is not additionally stressed by necessary retention elements. By virtue of the greater retaining effect associated with this, the surface of the implant particularly around the outlet region can be made larger, such that coverage by epithelial cells is prevented. The latter is undesirable particularly in the region of the openings of the implant, whereas a growth of the cells at the edge of the retention element, in particular on the eye surface, can be advantageous for smoothing the transition zone, in order to avoid or reduce the foreign-body sensation during eyelid closure.

In this context, it is likewise advantageous for example that the components of the shunt implant are present in different dimensions. This permits an exact adaptation to different thicknesses of cornea and sclera.

The thickness of the cornea or sclera can be measured for example by application of OCT or ultrasound technology, in order thereafter to adapt a corresponding shunt implant to the determined thickness.

According to a further example embodiment, at least one of the components of the shunt implant has a pressure-measuring means, which can preferably be read passively.

For passive reading, it is possible for example to use RFID (radio-frequency identification) technology, in which identification takes place with the aid of electromagnetic waves. In RFID systems, the code or measurement value contained in a transponder is read contactlessly by a reader.

It is moreover advantageous that the proposed invention is not limited just to single implants, and instead the inner component and outer component can also be designed such that double implants or multiple implants are produced.

For this purpose, FIG. 4 shows shunt implants according to the invention in the form of a single implant and a double implant. A double implant 8 (left-hand image) and a single implant 9 (right-hand image) are shown here, both anchored in the cornea 3.

While the inner components of the shunt implant are positioned individually by use of inserters for a micro-incision, the one outer component here is secured by a clip mechanism. Double or multiple implants permit a more reliable drainage action, through multiple parallel outlet channels, and they additionally afford greater stability.

With the solution according to the invention, a shunt implant for glaucoma treatment is made available which permits direct drainage through the cornea, the limbus or the sclera and which does not have the disadvantages of the solutions known in the prior art.

The particular configuration composed of at least two components allows the shunt implant, or also just components thereof, to be easily removed or replaced. The strain on the tissue is therefore extremely low, even during removal.

The enlarged inlet and outlet surfaces serve for a more reliable hold and less strain on the tissue, and they additionally reduce or prevent cell coverage, particularly in the inlet region and outlet region of the shunt implant.

The shunt implant can moreover be designed such at least one part can perform a cutting or piercing action that supports the insertion (for example a cutting edge or tip on the inner part), and, after connection to a further implant, this is deactivated or made safe, for example covered by the screwed-on outer part.

1.-19. (canceled)

20. A shunt implant for glaucoma treatment to facilitate direct drainage through the cornea, through the limbus or through the sclera onto the eye surface, the shunt implant comprising:
at least one inner component structured to be inserted and/or manipulated from inside the eye and one outer component structured to be inserted and/or manipulated from outside the eye, wherein the at least one inner component includes folding haptics, and the at least one inner component and the one outer component are connectable to each other following introduction.

21. The shunt implant as claimed in claim 20, wherein the cross section of the shunt implant is round, oval or else angular.

22. The shunt implant as claimed in claim 20, further comprising a tool to introduce the at least one inner component of the shunt implant wherein the tool comprises an inserter that facilitates insertion through a micro-incision.

23. The shunt implant as claimed in claim 20, wherein the at least one inner component of the shunt implant presents corresponding indentations structured to receive the folding haptics.

24. The shunt implant as claimed in claim 20, wherein the folding haptics of the at least one inner component of the shunt implant are structured such that a slight spring action is exerted between the shunt implant and tissue holding the shunt implant.

25. The shunt implant as claimed in claim 20, wherein parts of the inner component or of the outer component comprise a material permeable to aqueous humor or have corresponding pores/openings.

26. The shunt implant as claimed in claim 20, wherein the at least one inner component of the shunt implant comprises a pressure-reducing element.

27. The shunt implant as claimed in claim 26, wherein the pressure-reducing element is designed specific to the patient and/or designed to be exchangeable.

28. The shunt implant as claimed in claim 20, wherein the at least one inner component and outer component of the shunt implant are connected by a clip, a magnetic connection or by a screw thread.

29. The shunt implant as claimed in claim 28, wherein the connection between the at least one inner component and the at least one outer component of the shunt implant is designed to be reversible.

30. The shunt implant as claimed in claim 20, wherein the at least one inner component and the at least one outer component of the shunt implant are connectable by intraoperative welding or adhesive bonding.

31. The shunt implant as claimed in claim 20, wherein the components of the shunt implant are present in different dimensions and can be combined for adaptation to different thicknesses of cornea or sclera.

32. The shunt implant as claimed in claim 20, wherein at least one of the components of the shunt implant further comprises a pressure-measuring sensor.

33. The shunt implant as claimed in claim 32, wherein the pressure-measuring sensor present can be read passively.

34. The shunt implant as claimed in claim 20, wherein the at least one inner component and the at least one outer component are designed such that double implants or multiple implants can be produced.

35. The shunt implant as claimed in claim 20, wherein the at least one inner component and the at least one outer component are made of a porous material.

36. The shunt implant as claimed in claim 1, wherein the at least one inner component and the at least one outer component are made of a disinfecting material.

37. The shunt implant as claimed in claim 20, wherein the at least one inner component and the at least one outer component are made of a transparent material.

38. The shunt implant as claimed in claim 20, wherein the at least one inner component and the at least one outer component are structured such that at least one of the at least one inner component and the at least one outer component have a piercing or cutting portion and are suitable for generating a micro-incision.
